(19) United States
(12) Patent Application Publication
(10) Pub. No.: US 2008/0076969 A1
(43) Pub. Date: Mar. 27, 2008

(54) Methods for modifying control software of electronic medical devices

(76) Inventors: Ulrich Kraft, Hofheim (DE); Manfred Ebner, Oberursel (DE); Joseph A. McCluskey, Sharon, MA (US); Manfred Ohlenschlager, Rimbach (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/512,070

(22) Filed: Aug. 29, 2006

(51) Int. Cl. A61B 5/00

(52) U.S. Cl. 600/300

(57) ABSTRACT

The present invention provides methods for modifying, upgrading, or otherwise revising control software, instructions, or algorithms of an electronic medical device. A portable memory device, such as a Smart Card or the like is used to provide information for modifying the control software to the electronic medical device.

### TECHNICAL FIELD

The present invention relates to electronic medical devices that are controlled by software or programming instructions such as those used for detection of various analyte levels in physiological fluid samples. More particularly, the present invention relates to methods for modifying the control software of such electronic medical devices.

### BACKGROUND

Electronic medical devices that are used for the detection of various analyte levels in physiological fluid samples are well known and popular with consumers. For example, the concentration of an analyte in a variety of different physiological samples, such as urine, tears, saliva, and the like can be determined with these devices. One popular application is for determining the concentration of an analyte in interstitial fluid, blood or blood fractions, and more particularly in whole blood.

Electronic medical devices such as analyte monitors are typically software controlled and often include functionality in addition to the ability to measure analyte concentrations. For example, these devices are often capable of storing readings taken over time, performing analysis of such readings, operating in different languages and with customizable user interfaces, and communicating with other electronic medical devices, computers, networks, or the like. Often, an upgrade or modification to the control software can provide new features and functionality or improvements to existing functionality with the same hardware. Moreover, such modifications can be used to configure a medical device for use by a specific user such as to set a language for a user interface or the like.

The software of typical electronic medical devices is programmed into the memory of the device at the time of its manufacture and is typically not modifiable by a user. Because software modification or updating is impossible for many devices, the only way for a user to take advantage of new features is to acquire a new electronic device with the new features. For medical devices that can be modified or otherwise updated, the process is complex, slow, and usually requires special technical knowledge and equipment to perform.

### SUMMARY

The present invention thus provides methods for modifying, upgrading, or otherwise revising control software, instructions, or algorithms of an electronic medical device. In accordance with the present invention a portable memory device, such as a flash memory chip, a Smart Media Memory device, a Single Inline Memory Module (SIMM), an electrically erasable programmable memory card, a Smart Card, or the like is used to provide information for modifying the control software to the electronic medical device. Using such a memory device to facilitate a software modification allows for modifications to be performed much faster than can be achieved by a serial update. Modifications that can be performed include upgrades or revisions to software code, configuration of operating parameter(s), and/or addition of new features or functionalities, for example.

In an aspect of the present invention, a method for modifying control software of an electronic medical device is provided. The method preferably comprises the steps of: providing a peripheral medical device comprising control software; providing a remote controller capable of wirelessly communicating with the peripheral medical device at a communication frequency; providing a portable memory device comprising information for modifying the software code of the peripheral medical device; establishing communication between the memory card and the remote controller; wirelessly transferring at least a portion of the information for modifying the control software of the peripheral medical device from the remote controller to the peripheral medical device; and using at least a portion of the information for modifying the control software of the peripheral medical device to modify the control software of the peripheral medical device. In accordance with the present invention the information for modifying the control software of the peripheral medical device can also be used to modify the control software of the remote controller.

In another aspect of the present invention, a method for modifying control software of an electronic medical device is provided. The method preferably comprises the steps of: providing a peripheral medical device comprising control software; providing a remote controller comprising control software and capable of wirelessly communicating with the peripheral medical device; providing a non-volatile memory card comprising information for modifying the control software of the peripheral medical device and information for modifying the control software of the remote controller; establishing communication between the memory card and the remote controller; modifying the control software of the remote controller based on the information for modifying the user interface of the remote controller; wirelessly transferring at least a portion of the information for modifying the control software of the peripheral medical device from the remote controller to the peripheral medical device; and modifying the control software of the peripheral medical device based on the information for modifying the control software of the peripheral medical device.

### BRIEF DESCRIPTION OF THE DRAWINGS

These and other features, aspects, and advantages of the present invention will become better understood with regard to the following description, appended claims, and accompanying drawings where:

FIG. 1 is a perspective view of an exemplary software controlled medical device, illustrated as a remote controller for a peripheral medical device, the software of which can be modified in accordance with the present invention;

FIG. 2 is a schematic diagram showing certain functional aspects of the remote controller of FIG. 1;

FIG. 3 is a flow chart showing an exemplary method for modifying the control software of the remote controller of FIG. 1 in accordance with the present invention;

FIG. 4 schematic diagram of a system in accordance with the present invention comprising the remote controller of FIG. 1, a peripheral medical device, and a memory device that can be used to modify the control software of one or both of the remote controller and peripheral medical device in accordance with the present invention;

FIG. 5 is a flow chart showing an exemplary method for upgrading the peripheral medical device of FIG. 4 using the remote controller of FIG. 4 in accordance with the present invention; and

FIG. 6 is a flow chart showing an exemplary method for upgrading the peripheral medical device and remote controller of FIG. 4 using the remote controller in accordance with the present invention.

### DETAILED DESCRIPTION

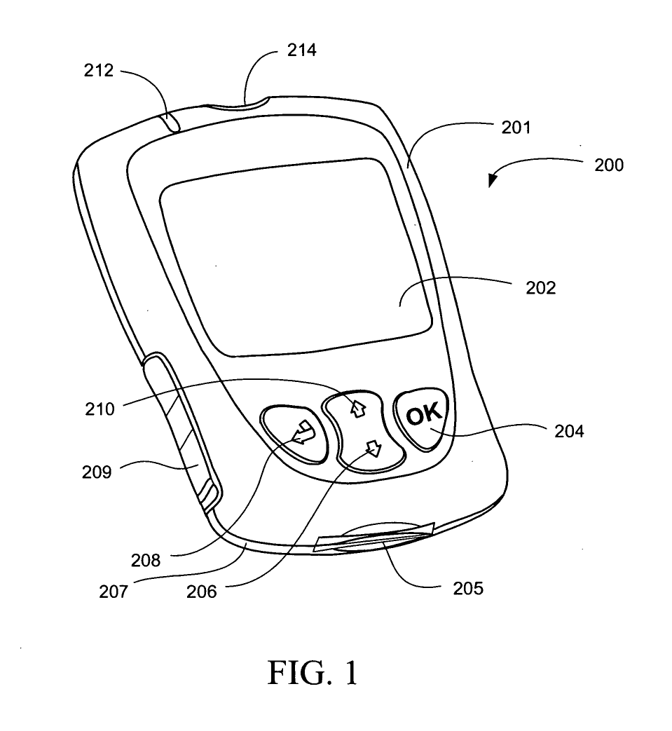

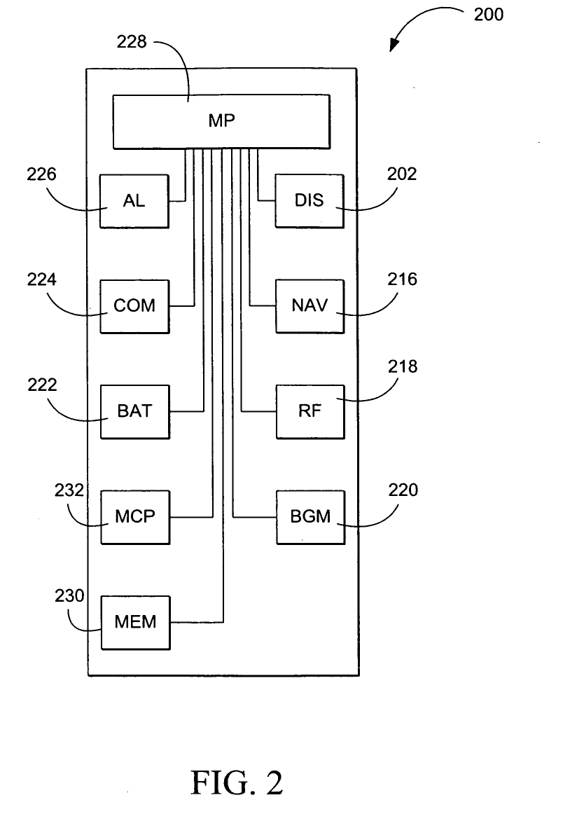

FIG. 1 is a perspective view of an exemplary medical device in accordance with the present invention, which as shown comprises a remote controller 200. The illustrated remote controller 200 functions to episodically measure blood glucose and can also wirelessly control an insulin pump, glucose monitor, or other similar device. Remote controller 200 includes a first housing portion 201, a display 202, an OK button 204, a universal port connector 205, a down button 206, a second housing portion 207, a back button 208, a port cover 209, an up button 210, light emitting diode 212, and a strip port connector 214. As illustrated, first housing portion 201 and second housing portion 207 join together to form an ergonomically shaped handheld device that incorporates functional circuitry for measuring glucose episodically and also for wireless communicating with an one or more additional peripheral medical devices. General functionality of remote controller 200 is schematically shown in FIG. 2 and schematically illustrates a display (DIS) 202, navigational buttons (NAV) 216, a radio frequency module (RF) 218, a blood glucose measurement (BGM) module 220, a battery (BAT) 222, a wired communication port (COM) 224, an alarm (AL) 226, a microprocessor (MP) 228, a memory (MEM) 230, and a memory chip port (MCP) 232.

Display 202 preferably comprises a liquid crystal display (LCD) to show both textual and graphical information to a user. A user interface for remote controller 200 preferably comprises a software driven menu that is viewable on display 202 and enables the user to operate remote controller 200. A user can navigate through the user interface using navigation buttons 216 which include up button 210, down button 206, OK button 204, and back button 208. The user interface allows a user to operate an insulin pump or other device, query the status of the insulin pump, measure glucose episodically, and display data on display 202 (e.g. glucose concentration versus time), for example.

Radio frequency module 218 of remote controller 200 provides for bi-directional communication with one or more peripheral medical devices such as a continuous glucose monitor, a personal computer, a personal digital assistant, a cell phone, insulin pen, or a pump. Exemplary frequencies which may be suitable for use with RF module 218 are about 433 MHz, about 863 MHz, about 903 MHz, and about 2.47 GHz. Radio frequency module preferably includes a microprocessor for managing a wireless signal, a transceiver, an antenna, and an antenna impedance matching network. RF module 218 preferably functions to send commands to or receive data from a peripheral medical device or the like. Such data can include information relating to status, operating conditions, or parameters of a peripheral medical device such as indicating an error condition, battery lifetime status, and historical information, for example.

BGM 220 preferably comprises a potentiostat designed for performing an electrochemical measurement of a glucose concentration. BGM 220 applies a constant potential such as, for example, about +0.4 V between a working electrode and a reference electrode of a disposable test strip. An exemplary disposable test strip is the OneTouch Ultra test strip which is commercially available from LifeScan, Inc. in Milpitas, Calif., U.S.A. In use, the disposable test strip is inserted into strip port connector 214. After insertion, a physiological fluid such as blood is applied to the disposable test strip causing the test to initiate. A reagent of the disposable test strip proportionally converts an oxidized mediator to a reduced mediator allowing a current to be measured. A portion of the current is sampled and mathematically converted to the glucose concentration, which is preferably displayed on display 202.

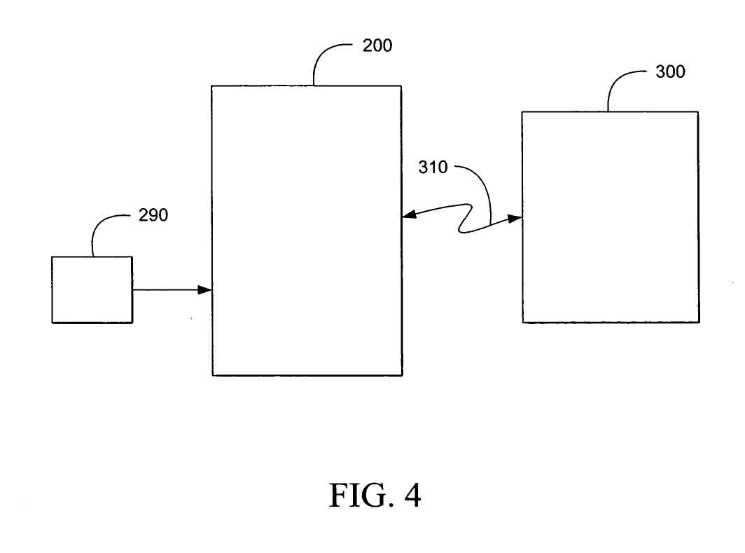

Port cover 209 preferably comprises an elastomeric material that covers over a wired connection port 224 and a memory chip port 232 for communication with a portable memory device such as a memory chip. Examples of a wired connection port include a universal serial bus (USB) or a serial RS232. As shown, memory chip port 232 comprises a slot in which the memory chip 290 can be inserted as shown in FIG. 4. Memory chip port 232 includes a connector having a plurality of contacts to electrically connect to memory chip 290. As an example, a Smart Card with an 8-pin connector can be used with a serial peripheral interface data transfer protocol.

In accordance with the present invention, the memory chip or memory device preferably comprises a portable memory device such as, for example, a flash memory chip, a Smart Media Memory device, a Single Inline Memory Module (SIMM), an electrically erasable programmable memory card, a Smart Card, or the like. In one aspect of the present invention, the memory device is used to provide information to remote controller 200 that can be used to modify control software of remote controller 200.

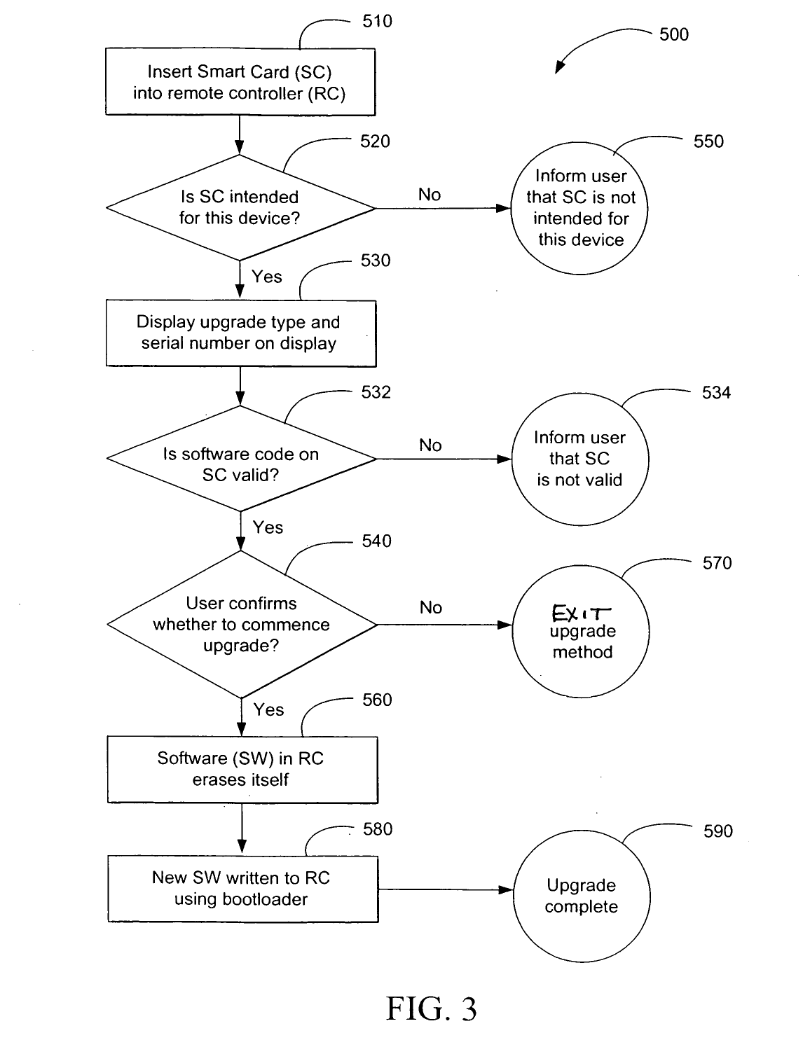

FIG. 3 shows a flow chart illustrating the steps of an exemplary method 500 of modifying remote controller 200 in accordance with the present invention. First a user establishes communication between the memory device and remote controller such as by inserting a portable memory device such as a Smart Card into memory chip port 232, as shown in step 510. Such communication can be established by direct, cable, or wireless connection. Upon establishing communication, remote controller 200 reads the portable memory device to determine that it is intended for use with remote controller 200, as shown in step 520. Memory chip 290 preferably comprises serial number information or the like to determine whether the modification is appropriate for remote controller 200. If remote controller 200 determines that the portable memory device was intended for use with remote controller 200, then remote controller 200 preferably shows the type of modification and version number on display 202, as shown in step 530. On the other hand, if remote controller 200 determines that the portable memory device was not intended for use with remote controller 200, then remote controller 200 preferably shows on display 202 that the portable memory device was not intended for this device, as shown in step 550.

After remote controller 200 displays the type of modification and version number, remote controller 200 preferably checks that the software code, instruction, or information on the portable memory device is valid, e.g. not corrupted, in step 532. If the information in the portable memory device is found to be valid, then the user is preferably prompted to confirm whether modification process 500 should be commenced, as shown in step 540. If the information in the portable memory device is found to be invalid, or corrupt, then remote controller 200 informs the user that the portable memory device is not valid on display 202, as shown in step 534.

If the information on the portable memory card is valid and the user confirms the commencement of the software modification, the software in memory 230 preferably erases itself as shown in step 560. If the user declines to modify the software, then remote controller 200 preferably notifies the user on display 202 that it is exiting the modification method 500, as shown in step 570. After erasing the software code in step 560, remote controller 200 then uses the information on the portable memory device to modify the remote controller 200 such as by writing software code from the portable memory device to memory 230 using a bootloader or the like, as shown in step 580. The bootloader preferably comprises a small program that loads an operating system into memory when the system is booted and also starts the operating system. The bootloader is preferably stored in a non-volatile portion of memory 230. After transferring the software code to memory 230, modification method 500 is complete as shown in step 590. Memory chip 290 preferably erases its software code after its transfer to memory 230 allowing memory chip 290 to be used only once.

Changes or modifications to a user interface are possible in accordance with the present invention. The user interface includes a set of menus through which the user communicates with remote controller 200 using navigation buttons 216. The user can select a command or input/edit data into remote controller 200. User interface modifications contemplated in accordance with the present invention include adding a menu selection that allows data to be processed and/or outputted in a new format such as, for example, in predicting a health index, a future glucose concentration, a hemoglobin AIC percentage, recommending an insulin bolus, or a basal rate. These types of outputs may be based on a plurality of episodic glucose measurements and/or continuous glucose measurements and the respective timing of these measurements, which may be collected by remote controller 200.

An operating parameter(s) of the remote controller 200 can also be modified in accordance with the present invention, such as, changing a first language to a second language on the user interface to accommodate a particular user for example. For example, a user may change the user interface language from English to German. Other operating parameters that can be modified or otherwise configured include a communication frequency for remote controller 200 such as changing first frequency to a second frequency of radio frequency module 218. This may be desirable because an Industry-Scientific-Medical (ISM) band typically ranges from about 868 MHz to about 869 MHz in Europe and typically ranges from about 902 MHz to about 928 MHz in the United States. Thus, a user or manufacturer of remote controller 200 may want to change the frequency of radio frequency module 218 depending on the location of use. For example, the first frequency may range from about 868 MHz to about 869 MHz and the second frequency may range from about 902 MHz to about 928 MHz. As another example, the first frequency may range from about 902 MHz to about 928 MHz and the second frequency may range from about 868 MHz to about 869 MHz.

Another example of an operating parameter that can be modified in accordance with the present invention relates to the glucose algorithm of remote controller 200. For example, in use, a glucose concentration can be determined by averaging a predetermined number of current measurements, which are then processed using a mathematical formula based on a calibration code. The predetermined number of current measurements or the mathematical formula or both can be changed in accordance with the present invention. In addition, the glucose algorithm may also include error codes capable of identifying routine mistakes by the user or defects in the glucose measuring system. Additional error codes can be added or alternatively error codes may be deleted or modified in accordance with the present invention.

Functionality can be added to and/or removed from remote controller 200 in accordance with the present invention. For example, a food database or the like can be added to the user interface of remote controller 200 or and existing food database can be updated or otherwise modified. The calculation of a food's carbohydrate content can be difficult to determine. The food database allows users to easily determine the carbohydrate content of their meal and thus facilitate the calculation of an insulin bolus before consuming food. Also, adding the ability of remote controller 200 to wirelessly communicate with one or more additional peripheral medical devices is also possible in accordance with the present invention. Remote controller 200 can also be modified to allow it to communicate with a different type of insulin pump such as, for example, an insulin pump from a different manufacturer.

The present method may also be used to modify or update a peripheral medical device by using a memory chip or device to provide information to the peripheral medical device to modify control software of the peripheral medical device. Such as is described above with regards to the remote controller 200, the exemplary modifications that can be made to remote controller 200 described above also apply to a peripheral medical device and vice versa. Communication between memory chip and remote controller is preferably provided by inserting memory chip 290 into memory chip port 232 of remote controller 200. Cable or wireless communication can also be used. Remote controller 200 preferably transmits a wireless signal 310 that transfers modified software code, instructions, information or the like to peripheral medical device 300 as shown in FIG. 4 and described in more detail below. Memory chip 290 is preferably removed from remote controller 200 before performing an episodic glucose measurement.

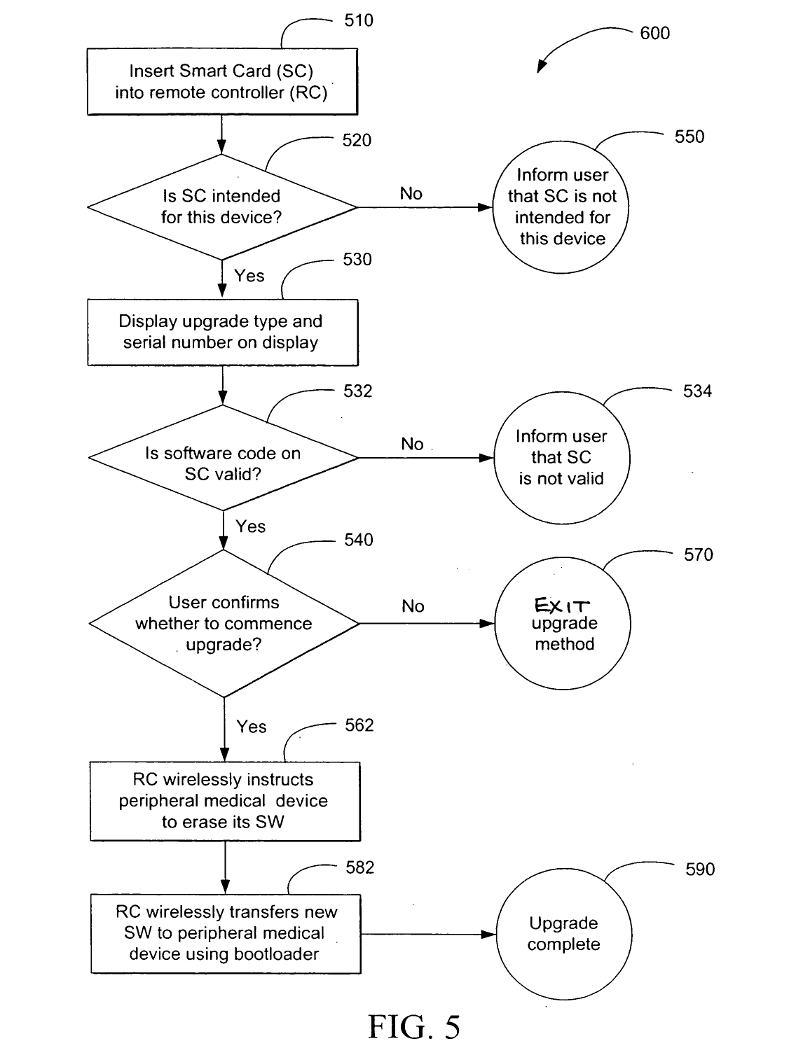

FIG. 5 is a flow chart showing exemplary method 600 for upgrading a peripheral medical device using remote controller 200 in accordance with the present invention. A user establishes communication between a memory device and the remote controller 200 such as by inserting a portable memory device such as, for example, a Smart Card or the like into memory chip port 232 of remote controller 200, as shown in step 510. Upon insertion, remote controller 200 preferably reads the portable memory device to determine that it is intended for a modification to the peripheral medical device, as shown in step 520. If remote controller 200 determines that the portable memory device was intended for the peripheral medical device, then remote controller 200 preferably shows the type of modification and version number on display 202, as shown in step 530. If remote controller 200 determines that the portable memory device was not intended for this device, then remote controller 200 preferably shows on display 202 that the portable memory device was not intended for this device, as shown in step 550.

After remote controller 200 displays the type of modification and version number, remote controller 200 preferably confirms that the software code, instruction, or information on the portable memory device is valid in step 532. This allows remote controller to determine that the portable memory device is not corrupted before starting the modification. If the software code in the portable memory device is found to be valid, then the user is preferably prompted to confirm whether modification process 500 should be commenced, as shown in step 540. If the software code in the portable memory device is not found to be valid, then remote controller preferably informs the user that the portable memory device is not valid on display 202, as shown in step 534 and the program may exit if desired. If the user confirms the commencement of the software modification, then remote controller 200 preferably wirelessly instructs the peripheral medical device to erase its software code as shown in step 562. If the user declines to modify the software, then remote controller 200 preferably notifies the user on display 202 that it is exiting modification method 600, as shown in step 570. After erasing the software code in step 562, remote controller 200 then preferably wirelessly transmits to the peripheral medical device the modified software code, instructions, information, or the like from portable memory device 290 to a memory portion of the peripheral medical device using a bootloader or the like, as shown in step 582. After transferring the modified software code to the peripheral medical device, modification method 600 is complete as shown in step 590.

The wireless protocol of the peripheral medical device can be modified or otherwise changed in accordance with the present invention. For example, the frequency of a wireless signal transmitted and received by the peripheral medical device can be modified and/or changed. A wireless handshake protocol between remote controller 200 and a peripheral medical device, a password for encryption for the wireless operation of the peripheral medical device, and/or a new power savings mode for increasing battery life can also be modified and/or changed in accordance with the present invention.

The glucose measuring method of a peripheral medical device, such as a glucose monitor, can also be modified and/or changed in accordance with the present invention. For example, a typical glucose monitor usually includes an electrochemical glucose biosensor, a pressure ring, a bolus detector, and a valve. Exemplary embodiments of a continuous glucose monitor may be found in U.S. patent application Ser. No. 10/811,446 entitled “Microfluidic Analytical System with Position Electrodes” filed on Mar. 26, 2004; U.S. patent application Ser. No. 11/193,656 entitled “Microfluidic System with Feedback Control, filed Jul. 28, 2005 and U.S. patent application Ser. No. 11/193,704 entitled “Method for Feedback Control of a Microfluidic System, filed Jul. 28, 2005, the disclosures of which are each fully incorporated by reference herein for all purposes. The electrochemical glucose biosensor comprises a working electrode coated with a reagent coating that proportionally generates reduced mediator in the presence of glucose. Typically, a voltage is applied to the working electrode which allows the reduced mediator to be measured as a current. An exemplary modification to the glucose measuring method includes one or more of changes to operating parameters such as a frequency of current sampling, a sampling time duration, an integration of current at a pre-determined time, an aggregate average of the current sampling, a magnitude of applied potential, and a frequency of an AC applied potential.

The pressure ring of such a glucose monitor typically comprises an annular shaped surface that presses against the skin to increase the rate of interstitial fluid flow through a concentrically positioned penetration member such as a lance or the like. The pressure ring can also be used to mitigate glucose concentration lag. An exemplary modification to the use of the pressure ring includes one or more of a change to operating parameters such as an amount of time programmed for 1) a retracted state (i.e. time that the pressure ring is up), 2) a deployed state (i.e. time that the pressure ring is down with pressure applied towards the user's skin layer), 3) an amount of pressure applied to the user's skin layer, and 4) a distance for urging the pressure ring into the user's skin layer.

The bolus detector typically includes one or more electrodes for determining the volume of interstitial fluid that has been withdrawn towards the glucose monitor or the rate of interstitial fluid efflux. Typically, a glucose measurement is performed after a certain amount of interstitial fluid has been collected. An exemplary modification to the bolus detector includes a change in timing for when a glucose measurement should be performed. The glucose measurement timing may be based on a pre-determined volume interval and/or a pre-determined rate. Under certain circumstances, a valve can be used to stop interstitial fluid efflux if a pre-determined rate or pre-determined volume has been achieved.

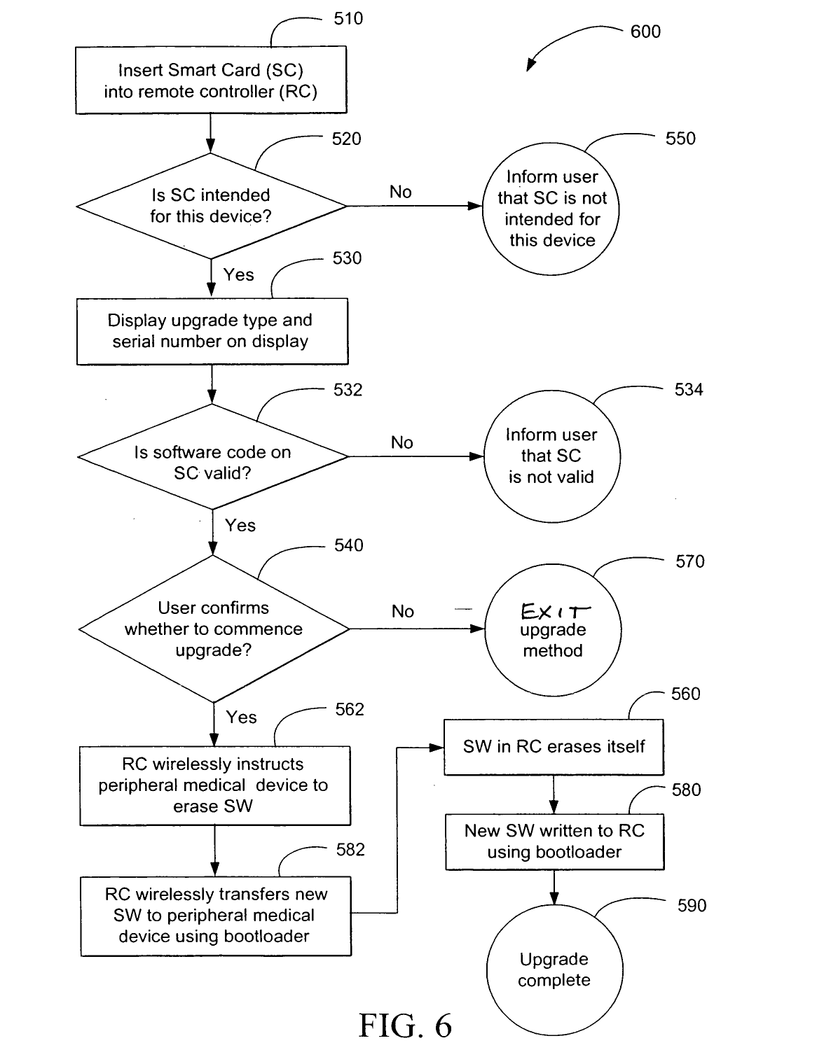

Remote controller 200 and a peripheral medical device can both be modified in accordance with the present invention by establishing communication between a portable memory device and remote controller 200. For example, a memory chip can be inserted into memory chip port 232. The remote controller 200 can transfer all or a portion of the information on the portable memory device (such as modified software code, instructions, or the like) to memory 230 of the remote controller and used to modify the remote controller 200. The remote controller can also wirelessly transfer all or a portion of the information on the portable memory device to the peripheral medical device as shown in FIG. 6. A modification of the peripheral medical device may also comprise a modification of the user interface of remote controller 200.

FIG. 6 is a flow chart showing an exemplary method 700 for modifying a peripheral medical device and remote controller 200 by using a portable memory device that can communicate with remote controller 200. A user establishes communication with a portable memory device such as by inserting a memory chip or the like into memory chip port 232 of remote controller 200, as shown in step 510. Upon insertion, remote controller 200 preferably reads the content of the portable memory device to determine that it is intended for a modification to the peripheral medical device and remote controller 200, as shown in step 520. If remote controller 200 determines that the portable memory device was intended for the peripheral medical device and remote controller 200, then remote controller 200 preferably shows the type of modification and version number on display 202, as shown in step 530. If remote controller 200 determines that the portable memory device was not intended for these devices, then remote controller 200 preferably shows on display 202 that the portable memory device was not intended for this device, as shown in step 550.

After remote controller 200 displays the type of modification and version number, remote controller 200 preferably confirms that the information on the portable memory device is valid in step 532. This allows remote controller to determine that the portable memory device is not corrupted before starting the modification. If the software code, instructions, information, or the like in the portable memory device is found to be valid, then the user is preferably prompted to confirm whether modification process 500 should be commenced, as shown in step 540. If the software code in the portable memory device is not found to be valid, then remote controller preferably informs the user that the portable memory device is not valid on display 202, as shown in step 534. If the user confirms the commencement of the software modification, then remote controller 200 preferably wirelessly instructs the peripheral medical device to erase its software code as shown in step 562. If the user declines to modify the software, then remote controller 200 preferably notifies the user on display 202 that it is exiting modification method 600, as shown in step 570. After erasing the software code in step 562, remote controller 200 then preferably wirelessly transmits to the peripheral medical device all or a portion of the modified software code from portable memory device 290 to a memory portion of the peripheral medical device using a bootloader or the like, as shown in step 582. Next, the software code in memory 230 preferably erases itself as shown in step 560. After erasing the software code in step 560, remote controller 200 then preferably writes all or a portion of the modified software code from the portable memory device to memory 230 using a bootloader or the like, as shown in step 580. After transferring all or a portion of the modified software code to the peripheral medical device and all or a portion of the modified software code to remote controller 200, modification method 700 is complete as shown in step 590.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

What is claimed is:

1. A method for providing information for modifying control software of a peripheral medical device from a remote controller to the peripheral medical device, the method comprising the steps of:
providing a peripheral medical device comprising control software;
providing a remote controller capable of wirelessly communicating with the peripheral medical device at a communication frequency;
providing a portable memory device comprising information for modifying the control software of the peripheral medical device;
establishing communication between the portable memory device and the remote controller; and
wirelessly transferring at least a portion of the information for modifying the control software of the peripheral medical device from the remote controller to the peripheral medical device.

2. The method of claim 1, comprising the step of using the information for modifying the control software of the peripheral medical device to change an operating parameter of the peripheral medical device.

3. The method of claim 2, wherein the operating parameter of the peripheral medical device comprises one or more of a language for a user interface and a communication frequency or protocol.

4. The method of claim 1, wherein the information for modifying the control software of the peripheral medical device comprises one or more of a software program or application, revised software code, instructions, and algorithms.

5. The method of claim 1, comprising using the information for modifying the control software of the peripheral medical device to change the communication frequency of the peripheral medical device from a first communication frequency to a second communication frequency.

6. The method of claim 5, wherein the first frequency ranges from about 868 MHz to about 869 MHz and the second frequency ranges from about 902 MHz to about 928 MHz.

7. The method of claim 5, wherein the first frequency ranges from about 902 MHz to about 928 MHz the second frequency ranges from about 868 MHz to about 869 MHz.

8. The method of claim 1, wherein the peripheral medical device comprises one of a continuous glucose monitor, an insulin pump, and an insulin pen.

9. The method of claim 1, wherein the remote controller is capable of measuring glucose episodically.

10. The method of claim 1, wherein the portable memory device comprises one or more of a flash memory card a flash memory chip, a Smart Media Memory device, a Single Inline Memory Module, an electrically erasable programmable memory card, and a Smart Card.

11. The method of claim 1, wherein the step of establishing communication between the portable memory device and the remote controller comprises physically connecting the portable memory device to the remote controller.

12. A method for providing information for modifying control software of a peripheral medical device from a remote controller to the peripheral medical device, the method comprising the steps of:
providing a peripheral medical device comprising control software;
providing a remote controller comprising control software and capable of wirelessly communicating with the peripheral medical device;
providing a portable memory device comprising information for modifying the control software of the peripheral medical device and information for modifying the control software of the remote controller;
establishing communication between the portable memory device and the remote controller;
modifying the control software of the remote controller based on the information for modifying the control software of the remote controller;
wirelessly transferring at least a portion of the information for modifying the control software of the peripheral medical device from the remote controller to the peripheral medical device; and
modifying the control software of the peripheral medical device based on the information for modifying the control software of the peripheral medical device.

13. The method of claim 12, comprising writing the information for modifying the control software of the peripheral medical device to a memory portion of the remote controller.

14. The method of claim 12, wherein the step of modifying the control software of the remote controller comprises modifying a user interface of the remote controller.

15. The method of claim 14, wherein the step of modifying the user interface of the remote controller comprises changing the language of the user interface from a first language to a second language.

16. The method of claim 12, wherein the step of modifying the control software of the remote controller comprises the step of providing or modifying a food database for estimating an amount of carbohydrate to the user interface of the remote controller.

17. The method of claim 12, wherein the peripheral medical device comprises one of a continuous glucose monitor, an insulin pump, and an insulin pen.

18. The method of claim 12, wherein the remote controller is capable of measuring glucose episodically.

19. The method of claim 12, wherein the portable memory device comprises one or more of a flash memory card a flash memory chip, a Smart Media Memory device, a Single Inline Memory Module, an electrically erasable programmable memory card, and a Smart Card.

20. The method of claim 12, wherein the step of establishing communication between the portable memory device and remote controller comprises physically connecting the portable memory device to the remote controller.
